(19) United States
(12) Patent Application Publication
(10) Pub. No.: US 2018/0271466 A1
(43) Pub. Date: Sep. 27, 2018

(54) MIXED MODE IMAGE GENERATION

(71) Applicant: Dieter Krotz, Erlangen (DE)

(72) Inventor: Dieter Krotz, Erlangen (DE)

(21) Appl. No.: 15/466,545

(22) Filed: Mar. 22, 2017

(51) Int. Cl. A61B 6/00; A61B 5/055; A61B 6/03; G06T 11/00

(52) U.S. Cl. CPC A61B 6/502; A61B 5/055; G06T 2210/41; A61B 6/482; G06T 11/003; A61B 6/032

(57) ABSTRACT

In order to reduce the time between a first computing device requesting display of an image and a second computing device, in communication with the first computing device, generating the image from medical scan data and sending the requested image to the first computing device, the medical scan data needed to generate the requested image is sent to the first computing device by the second computing device prior to the request so that the image may be generated locally at the first computing device.

### FIELD

The present embodiments relate to the generation of images from medical scan data.

### BACKGROUND

A client may request to view images (e.g. mammography or ultrasound images) by sending a request (e.g. initiated by user input such as a mouse click) through a network to a server. When the server receives the request, the server generates the requested images from corresponding medical scan data stored on the server (e.g. stored according to the Digital Imaging and Communications in Medicine (DICOM) standard) and sends the images to the client. The bandwidth of the connection between the client and server may not quickly transmit large image files (e.g. over 2 MB) generated for high resolution monitors (e.g. over 2 megapixels), resulting in delay between the time that the request is sent and the image is received by the client. Slow transfer may cause low viewing performance for the client.

### SUMMARY

In order to reduce the time between the client requesting display of an image and a server generating the image from corresponding medical scan data and sending the requested image to the client, the medical scan data for generation of the requested image is sent to the client by the server prior to receiving the request to display the image. The client generates the image from the medical scan data locally. The client may also be configured to receive images generated from medical scan data by the server. The server decides which medical scan data is sent to the client (without the server using the data to generate images), and which medical scan data is to be used by the server to generate images to be sent to the client. The decision may be based on a property of the medical scan data, the hardware and/or software configuration of the client, or a property of the connection between the client and server. For particular types of medical scan data (e.g. high resolution scans), the server may decide to send the medical scan data to a first subset of clients (e.g. clients that have high resolution monitors and meet or surpass minimum hardware or software requirements to generate images locally) and send the images generated from the medical scan data to a second subset of clients (e.g. those clients that do not meet the minimum requirements to generate images locally).

In a first aspect, a medical scan data system includes a first computing device in communication with a plurality of second computing devices. The first computing device includes a memory configured to store medical scan data and a processor in communication with the memory. The processor is configured to identify a first subset of the medical scan data and a second subset of the medical scan data, to identify a second computing device of the plurality of computing devices based on the identified first subset of the medical scan data, and to generate an image based on the second subset of the medical scan data. The processor is also configured to send the identified first subset of the medical scan data to the identified second computing device and to send the generated image to another second computing device of the plurality of second computing devices.

In a second aspect, a method of generating images from medical scan data includes identifying, by a processor in communication with a plurality of computing devices, a first subset of the medical scan data and a second subset of the medical scan data. The processor identifies a first computing device of the plurality of computing devices based on the identified first subset of the medical scan data and generates an image based on the second subset of the medical scan data. The processor also sends the identified first subset of the medical scan data to the identified first computing device, and sends the generated image to a second computing device of the plurality of computing devices.

In a third aspect, an apparatus for generating images from medical scan data includes a first computing device in communication with a second computing device. The first computing device includes a memory configured to store first medical scan data and a processor in communication with the memory. The processor is configured to send an identification to the second computing device; to receive the first medical scan data from the second computing device based on the identification, and to receive a request to display a first image and a second image. The first image corresponds to the first medical scan data received by the first computing device and the second image corresponds to second medical scan data stored at the second computing device. The processor is also configured to receive the second image from the second computing device, to generate the first image from the first medical scan data, and to display the first image and the second image.

In a fourth aspect, a method of generating images from medical scan data includes sending, by a processor in communication with a computing device, an identification to the computing device and receiving first medical scan data from the computing device based on the identification. The processor receives a request to display a first image and a second image. The first image corresponds to the first medical scan data received by the first computing device and the second image corresponds to second medical scan data stored on the second computing device from the medical scan data. The processor receives the second image from the computing device, generates the first image from the first medical scan data, and displays the first image and the second image.

### BRIEF DESCRIPTION OF THE DRAWINGS

FIG. 1 shows one embodiment of an image generation system;

FIG. 2 shows a flowchart of an example of a method for generating an image; and

FIG. 3 shows a flowchart of an example of a method for displaying medical scan data.

### DETAILED DESCRIPTION OF THE DRAWINGS

An additional medical imaging backend is provided on a client computer to generate images locally from medical scan data stored on the client, improving performance over transmitting to the client the images generated by a server-based medical imaging backend in response to a request to view the images. Client-based image generation may be used in conjunction with server based image generation, for example, such that images with a large network impact are generated on the client, whereas images with a lesser network impact are generated on the server and sent to the client for display. Clients without a local imaging backend may still receive images generated on the server.

The additional medical imaging backend may implement a DICOM image cache. The DICOM image cache may be filled automatically from the server side or only at request from the client. The additional medical imaging backend may include, for example, 500 MB or more of additional RAM for the additional processing. A viewer frontend at the client is connected with two viewer backend processes; for example, based on data availability at the client, the viewer frontend works with the additional medical imaging backend at the client computer or a viewer backend at the server.

For the client-based image generation, mouse events are processed locally on the client, and mouse events may not be transferred to the server. The client-based image generation may only be done for specific types of images. Data for the specific types of images may be automatically routed to specific clients before the corresponding case is opened at the clients.

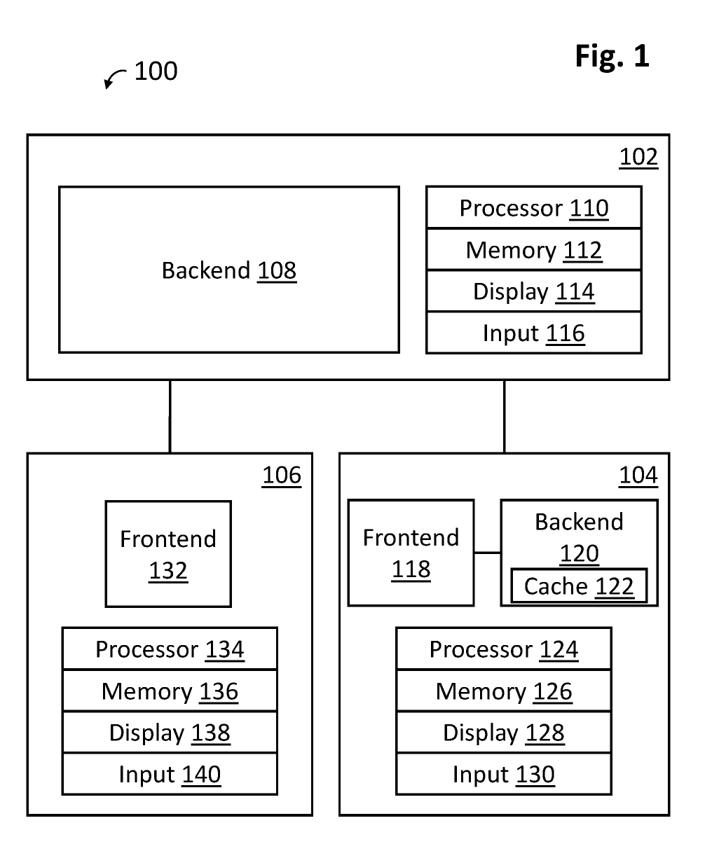

FIG. 1 shows one embodiment of an image generation system 100. The system 100 implements methods described below or other methods.

The system 100 includes a first computing device 102 (e.g. a server) and a plurality of second computing devices 104, 106 (e.g. clients). Additional, different, or fewer components may be provided. For example, there may be multiple servers operating independently or in a load balancing configuration. As another example, one or more computer networks with wireless and/or wired communications connections connect to interface cards on the server and clients.

The server 102 includes a backend 108. The server may also include a processor 110, memory 112, a display 114, and an input device 116. Additional, different, or fewer components may be provided. For example, the server may lack a display 114 and input device 116, or may have multiple processors 110.

The memory 112 of the server stores a set of medical scan data. In some cases, the medical scan data is generated by a medical scan of a subject and represents the subject. The data may represent more than one subject or be generated by multiple imaging devices or modalities.

The server 102 may be in communication with the clients 104, 106, for example, by a communication line, a cable, a wireless device, a communication circuit, a network, a communication device, or any combination thereof. In some cases, the server 102 is connected to imaging devices or other clients. For example, the server 102 may be in communication with a MR, CT, or other imaging device.

The backend 108 may be configured to receive medical scan data from one or more imaging devices, to generate images from the medical scan data, and to send the medical scan data and images to the clients 104, 106. In some cases, the backend 108 stores the medical scan data in the memory 112 according to the DICOM standard. Additionally or alternatively, the backend 108 stores the medical scan data in storage in communication with the server 102. The backend generates images from the medical scan data with the processor 110 or a processor in communication with the server 102.

The backend 108 may be further configured to send images and/or data to one or more clients. Different combinations of data and images may be sent to any given client. Data only may be sent to one client, the image only may be sent to another client, or both the data and the image may be sent to yet another client. For example, the server 102 sends images to one client (e.g. 106) and sends both images and medical scan data to a different client (e.g. 104).

For example, the backend 108 may be configured to send just medical scan data, but not images, to a client (e.g. for a client entering “offline mode” or for a client that will generate images from the medical scan data locally).

In some cases, the backend 108 forms part of a multimodality reading solution. For example, the backend 108 may form part of a multimodality reading solution incorporating CT, MR, PET, SPECT, mammography, Ultrasound, Angiography, Radiography, and Fluoroscopy imaging modalities, such as syngo.via from Siemens Healthcare GmbH.

Additionally or alternatively, the backend 108 may form part of a mixed-mode rendering image generation system that supports image generation on servers (e.g. 102) as well as clients (104, 106). The mixed-mode rendering image generation system is based, for example, on syngo.via or another reading solution.

The client 104 is in communication with the server. For example, a client may be a general-purpose computer or a specialized medical imaging workstation. The client is configured for local generation of images from medical scan data.

The client 104 may present identification to the server 102. For example, the client identification may contain information about the name, location, hardware capabilities, software configuration of the client. Additionally or alternatively, the identification may contain an index for a table accessible by the server 102.

The client 104 may include a frontend 118 and a local backend 120. The local backend 120 may include a cache 122. The client may further include a processor 124, memory 126, a display 128, and an input device 130.

The frontend 118 may be in communication with one or more backends 108, 120. In some cases, the frontend 118 may be connected to the local backend 120 and the server backend 108. Additionally or alternatively, the frontend 118 may be connected to the local backend processes of other clients.

The frontend 118 receives requests for display of images, for example, from the input device 130 for viewing on the display 128. Additionally or alternatively, the request may come from another client (e.g. 106)

The frontend 118 presents images generated from medical scan data present at the client (e.g. 104, 106) or server (e.g. 102). In some cases, the images are generated by the local backend 120 of the client 104, with medical scan data present at the local backend 120, the memory 126, or otherwise at the client 104.

The medical scan data stored on the server 102 may be transferred to the client 104 prior to the request to display an image in order to reduce any delay caused by the network at the time the request is received by the frontend 118. The server 102 may send medical scan data to the client based on the client identification, the medical scan data present at the server 102, historical requests for medical scan data fulfilled by the server 102, network information, or any combination thereof.

For example, the server 102 sends medical scan data generated by a mammography imaging modality to a client that has identified itself as a mammography workstation with a high-resolution monitor and at least the minimum hardware and software configuration necessary for local generation of images from mammography data. Images may then be generated by the client 104 (e.g. by the backend 120) using the local medical scan data instead of the medical scan data stored at the server 102, eliminating the need to send requests for images to be generated by the server 102 for medical scan data already present at the client 104.

The local backend 120 receives medical scan data and generates images from the medical scan data. The local backend 120 may include similar, additional, or fewer components than the backend 108. For example, the backend 120 may not include data or workflow management services present in the server backend 108. Additionally or alternatively, the local backend 120 may be implemented by the processor 124 or by a processor in communication with the client 104.

Additionally or alternatively, the frontend 118 may receive a request for an image generated from medical scan data not stored at the client 104. For example, the frontend 118 receives a request to view an image that is generated from medical scan data on the server 102 (e.g. by the backend 108), and the server 102 sends the client 104 the generated image but not the medical scan data used to generate the image.

In some cases, a particular medical case or medical scan data generated by a particular imaging modality may be selected for local image generation at the client 104 by the local backend 120. For example, the server 102 or a user at the client 104 may select mammography images to be at least partially generated on the client 104 (e.g. within a graphical user interface (GUI)), and the server 102 sends the respective medical scan data to the client 104.

The medical scan data sent to the client 104 for local image generation by the backend 120 may be determined automatically. For example, images where a product of the image frame size and resolution of the display 128 exceeds a threshold may be automatically selected for image generation at the client 104 and sent to the client 104.

Medical scan data may be withheld from the client 104 (e.g. residing on the server 102) based on certain criteria. For example, computed tomography (CT) or magnetic resonance imaging (MRI) data may be selected for image generation at the server 102 because images generated from medical scan data from such modalities do not result in large image files. Additionally or alternatively, the server 102 may not send the medical scan data to the client 104 where the display 128 of the client 104 has a low resolution.

Other applications may access the medical scan data stored by the local backend 120. For example, an application running on the client 104 or 106 may access the medical scan data stored by the backend 120. For example, additional programs (e.g. beyond the frontend 118) may be implemented by the processor 124 and access DICOM data stored in the cache 122 of the backend 122. The local backend 120 may generate and send images to the frontend 118 or to another client 106.

The cache 122 is a memory for storing the medical scan data. In some cases, the cache 122 is a DICOM image cache. The data may be sent to the cache 122 automatically or at the request of the client. For example, medical scan data of a certain type or having a certain attribute may be sent from the server 102 to the client 104. In a further example, the data is sent to the client 104 because a particular medical case or class of medical data has been frequently opened at the client 104. In another example, the cache stores medical scan data according to a worklist for the user that specifies medical cases of imaging modalities the user will work on.

The cache 122 may be present in the memory 126 or in a memory in communication with the client 104. For example, the cache 122, or the backend 120 as a whole, may reserve 500 MB of space in the memory 126. The cache 122 may store medical scan data and provide the medical scan data to the backend 120 to generate images from the medical scan data.

The processor 124 may display the images on the display 128. In some cases, the processor 124 generates the image from medical scan data in the cache 122 and communicates the image to the display 128.

The display 128 is a monitor, a CRT, an LCD, a plasma screen, a flat panel, a projector, or another now known or later developed display device. For example, the display 128 may be a high-resolution monitor suitable for viewing mammography or ultrasound images.

The memory 126 is a computer readable storage media (e.g., non-transitory computer-readable storage media). The computer readable storage media may include various types of volatile and non-volatile storage media, including but not limited to random access memory, read-only memory, programmable read-only memory, electrically programmable read-only memory, electrically erasable read-only memory, flash memory, magnetic tape or disk, optical media, and the like. The memory 126 may be a single device or a combination of devices. The memory 126 may be adjacent to, part of, networked with, and/or remote from the processor 124.

The input 130 may be, for example, a keyboard, mouse, trackball, drawing tablet, or touchscreen. Other input devices may be provided.

A second client 106 may be in communication with the server 102. The server 102 may be in communication with additional or fewer devices than shown in FIG. 1.

The client 106 may include a frontend 132. The client 106 may also include a processor 134, memory 136, a display 138, and an input 140. The processor 134, memory 136, display 138, and input 140 of the client 106 may be the same or similar to the processor 124, memory 126, display 128, and input 130 of the client 104 or the processor 110, memory 112, display 114, and input 116 of the server 102, respectively. All the clients 104, 106 in communication with the server 102 may have respective frontends 118, 132, but none, some, or all of the clients 104, 106 in communication with the server 102 may have a respective backend 120.

In some cases, the client 106 is configured to receive images from the server 102. The received images may be stored in the memory 136 and displayed by the frontend 132 on the display 138. The frontend 132 may be implemented by the processor 134 or by a processor in communication with the client 106.

The frontend 132 may be configured to display the images generated from the medical scan data and sent to the client 106 by the server 102. In some cases, the frontend 132 is not in communication with a local backend (e.g. 120) of the client 106 and displays images generated by the server backend 108. Additionally or alternatively, the frontend 132 may be configured to display images generated at one or more server backends 108 or one or more local backends 120 of other clients 104.

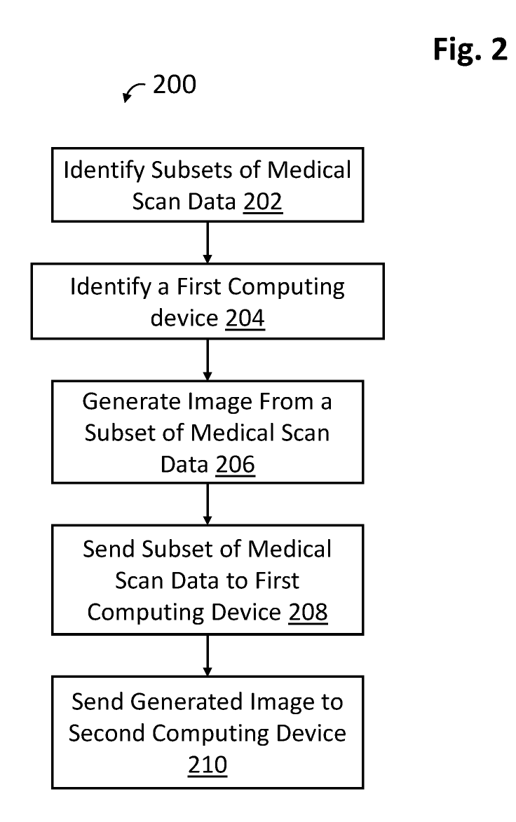

FIG. 2 shows a flowchart of one embodiment of a method 200 for generating an image from medical scan data for display. The scan data may, for example, be CT scan data or scan data generated during rotation of a C-arm during X-ray imaging. Alternatively, the scan data may be generated with another imaging device such as, for example, an MRI device.

The method 200 is performed by a first computing device (e.g. a server) in communication with a plurality of second computing devices (e.g. one or more of clients 104, 106). The method may be performed using the image generation system 100 shown in FIG. 1 (e.g., at least some of the acts of the method may be performed by the processor 110) or another system. For example, the acts of the method are implemented by one or more processors using instructions from one or more memories.

In act 202, a first subset and a second subset of the medical scan data are identified. The subsets may be identified, for example, by a processor of the server (e.g. 110). The subset may be identified based on the type of medical scan data, the imaging device with which the data was generated, the content of the medical scan data (e.g. a particular subject or anatomical part of a subject), properties of the medical scan data (e.g. file size, resolution, bit depth, or compressibility), the case that the medical scan data corresponds to, or any combination thereof.

In some cases, the first subset of medical imaging data is of a different type than the second subset of medical imaging data. For example, the first subset of medical imaging data may be mammography or ultrasound data, and the second subset of medical imaging data may be data that is not mammography or ultrasound data. For a further example, the first subset of the medical scan data is mammography scan data, and the second subset of the medical scan data includes scan data generated by a CT device, scan data generated by an MRI device, scan data generated by an X-ray device, or any combination thereof. In some cases, the medical scan data in the first and second subset of the medical scan data is a DICOM image. Other formats may be used.

The medical scan data, including the first and second subsets of the medical scan data, may be stored at the server. In some cases, the medical scan data and subsets are stored in the memory 112. Additionally or alternatively, the medical scan data, including the first and second subsets of the medical scan data, may be stored in memory in communication with the server.

In act 204, a first client in communication with the server is identified. In some cases, the first client is identified based on the first subset of the medical scan data. For example, the first subset of the medical scan data may be mammography data, and the server may identify a first client according to minimum system requirements for generating images from mammography data locally on the client. A client that does not meet or exceed the minimum requirements for generating images from mammography data locally would not be identified as the first client.

Alternatively or additionally, the first client may be identified based on the properties of the communication link between the server and the clients (e.g. speed, bandwidth, latency, or reliability), the configuration of the clients (e.g. processing power, graphics capability, installed or available memory, storage capacity, monitor resolution, speed of a network interface card, identification number, other hardware information, or location), the identified subsets of medical scan data, or any combination thereof. For example, a first client may be identified based on both the subset of medical scan data identified in step 202 as containing mammography scan data and the client having a high-resolution display suitable for viewing mammography images.

In some cases, the first client is identified automatically by the server or based on user input. For example, the server may automatically identify the client device using any one or more of the above criteria.

Alternatively or additionally, the first client is identified based on a setting selected by a user for local generation of images at the client from all or a subset of the medical scan data. For example, when the user selects that a list of medical cases opened at the client are to have at least some images generated locally, the server may send the client the medical scan data for those cases (e.g. the client is identified as the first client in act 204).

In act 206, an image is generated from the second subset of the medical scan data. In some cases, the image is generated by a backend (e.g. 108) of the server. For example, the server backend may generate the image from DICOM medical scan data stored in the server memory 112. In a further example, a syngo.via backend on the server generates the image from the medical scan data.

In act 208, the first subset of the medical scan data is sent to the first client. The first subset of the medical scan data may be sent by the processor of the server (e.g. processor 110).

The medical scan data may be sent to the first client so that the first client may later generate the image from the medical scan data locally (e.g. with a local backend 120) instead of sending a request to display an image to the server across the network, having the server generate the image from the medical scan data, and having the server send the generated image back across the network to the first client. Because the medical scan data is already present at the client prior to the generation of a request to view images (e.g. by the user), images may be generated from the medical scan data by the first client locally, reducing the impact of the network on the perceived performance of the first client in fulfilling requests.

Additionally or alternatively, medical scan data may be sent to the identified first client in response to medical scan data being received at the server. In other words, after the server receives medical scan data from an imaging device, the server may then automatically send the medical scan data to the identified first client. The automatic sending may be based on, for example, a filled checkbox in the GUI of the client or an identification of a display at the client Alternatively or additionally, the automatic sending may be based on the imaging modality that generated the medical scan data, the file size of the medical scan data, or the file size of the image to be generated from the medical scan data.

The first client may generate the image from the medical scan data sent by the server. For example, the medical scan data may be stored in a cache of a local backend on the client (e.g. 120, 122) and then used by a processor (e.g. 124) to generate the image for viewing (e.g. by the frontend 118 on display 128).

In act 210, the image generated in step 206 is sent to a second client in communication with the server. The generated image may be sent before or after the medical scan data is sent to the identified first client in step 208.

The second identified client may be configured to receive only images generated at the server, or may be configured to receive both images and medical scan data. For example, the second client may use outdated hardware (e.g. a slow processor, insufficient memory, or a low-resolution display) or software (e.g. an older operating system, drivers, or image viewing frontend) unsuitable for local generation of images from medical scan data.

In a further example, the second client has adequate hardware for local image generation, but the type of medical scan data produces images that have a small file size. A small file size may mitigate any perceived reduced performance at the client due to the network. Additionally or alternatively, the second client and the server may share a robust connection (e.g. high speed, low latency, or high reliability) that allows for the server to generate and send even large image files across the network without poor performance.

Alternatively or additionally, the image generated in step 206 may also be sent to the identified first client in communication with the server. In this way, the first client may be configured to receive both images and medical scan data. For example, the first client may generate mammography images locally due to the large image file size, but still receive pre-generated CT images from the server with a small image file size.

The method is implemented in the order shown, but other orders may be used. For example, the subset of medical scan data may be sent at the same time or before the image is generated from the second subset of medical scan data. In another example, the generated image is sent before the subset of medical scan data is sent.

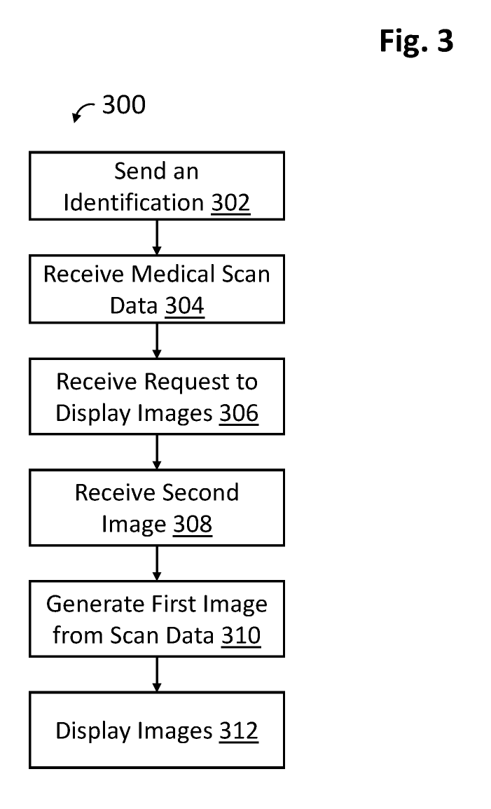

FIG. 3 shows a flowchart of one embodiment of a method 300 for generating an image for display. The method 300 may be performed, for example, by a computing device described above (e.g. the client 104 in FIG. 1). The client or a processor of the client performing the method may be in communication with a second computing device (e.g. the server 102 in FIG. 1).

In some cases, the server may be configured to perform the method 200 of FIG. 2. For example, the server provides a syngo.via backend for the frontend of the client.

In act 302, the client sends an identification to the server. The identification may contain information about the configuration of the client. For example, the identification may contain hardware specifications such as the processor speed and architecture, total and available memory, the presence and status of a local backend running on the processor, and the speed of a network interface card in communication with the processor. In a further example, the identification contains information about the configuration of a viewing frontend running on the processor and whether or not the user has selected the client for local generation of images from medical scan data. In a further example, the identification contains an index to a table, stored on the server, an entry of which contains more information about the processor or the client. In a still further example, the identification contains information about the configuration of the client, such as the resolution of a display of the client.

The identification may be used by the server to determine which medical scan data is appropriate to be sent to the client for local generation of images from the medical scan data. Additionally or alternatively, the server uses the identification along with properties of the medical scan data to determine which medical scan data to send.

In act 304, the client receives first medical scan data from the server based on the identification in act 302. For example, the medical scan data may be formatted according to the DICOM standard.

In some cases, the received first medical scan data is stored in the memory of the client (e.g. memory 126). Alternatively or additionally, the medical scan data may be stored in a cache of a local backend on the client (e.g. backend 120 with cache 122).

In some cases, the medical scan data is received by the client prior to receiving a request to display an image generated from the medical scan data. Receiving the medical scan data before the request allows for the local generation of images by the client and mitigates any reduced performance caused by the server generating and sending a large image file to the client after the client receives a request to display the image.

In act 306, a request to display a first image and a second image is received. The first image corresponds to the first medical scan data received in act 304, and the second image corresponds to second medical scan data stored at the server. For example, the client receives a request from a user to display images that need to be generated from medical scan data, and part of the medical scan data is stored locally on the client, while another part of the medical scan data is stored at the server.

The client may receive one or more requests to display the first image and the second image. For example, the user may cause one request to be generated to view both the first image and the second image (e.g. by opening a medical case in the frontend). Additionally or alternatively, the user may cause separate requests to be generated. For example, the user causes at least one request to be generated to display the first image, and at least a second request to be generated to display the second image (e.g. by opening separate medical cases on the client).

The generation of a request may be initiated by the user. In some cases, user input (e.g. a mouse click of the user) may initiate generation of a request to view an image (e.g. through the GUI of the frontend 118 of FIG. 1). For example, the user interacting with a GUI on the client may attempt to view a 2-dimensional image of a 3-dimensional representation of a subject (e.g. as represented by the medical scan data). In response to the attempt, the client generates a request to display the image, which can be fulfilled by a backend (e.g. of the client or server) by generating the image from the medical image data.

In some cases, a first mouse click may be processed by the client to generate a request that is fulfilled by the client locally (e.g. instead of sending the request to be fulfilled by the server) to generate a first request to view a first image that may be generated from medical scan data stored on the client. The client generates the first image to fulfill the first request. In a further example, a second mouse click may be processed by the client to generate a second request to view an image that may be generated from medical scan data not stored on the client. The client forwards the second request to the server for fulfillment.

In act 308, the second image is received from the server. The second image may be received in response to the client forwarding the request to display the second image to the server.

The server may generate the second image from the second medical scan data by the server backend. Additionally or alternatively, the second image may be generated from the second medical scan data by a backend of another client computer with a local backend.

In act 310, the client generates the first image from the first medical scan data. In some cases, the image is generated by the local backend (e.g. 120) or processor (e.g. 124) of the client.

Generation of the image by the local backend may allow for image generation without sending the request to view the image to the server. Local image generation improves the perceived performance of the client because the client avoids waiting for the request to be sent to the server through the network, for the server to generate the image from medical scan data, and for the server to send the image back across the network. Instead, the request to view the first image is fulfilled locally when the client generates the first image from the first medical scan data already present on the client.

In act 312, the first image and the second image are displayed by the client. In some cases, the images are displayed by a frontend running on a processor of the client (e.g. frontend 118 implemented by the processor 124).

The first image and the second image may be displayed on a display of the client (e.g. 128) or a display in communication with the client. The first image and the second image may be displayed at the same or at different times at the client.

The client may be configured to receive images generated at the server and display them in a similar or the same way as images generated by the client. Alternatively or additionally, the first subset and the second subset of the medical scan data may contain different types of medical scan data. For example, the first subset of the medical scan data may be mammography scan data, and the second subset of the medical scan data may include scan data generated by a CT device, scan data generated by an MRI device, scan data generated by an X-ray device, or any combination thereof.

While reference has been made above to medical scan data, the scan data may be non-medical in nature. For example, the scan data could be generated by an optical scanner and represent a 3-dimensional object such as a machine.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A medical scan data system comprising:
a first computing device in communication with a plurality of second computing devices, the first computing device comprising:
a memory configured to store medical scan data; and
a processor in communication with the memory, the processor being configured to:
identify a first subset of the medical scan data and a second subset of the medical scan data;
identify a second computing device of the plurality of computing devices based on the identified first subset of the medical scan data;
generate an image based on the second subset of the medical scan data; and
send the identified first subset of the medical scan data to the identified second computing device, and send the generated image to another second computing device of the plurality of second computing devices.

2. The medical scan data system of claim 1, wherein the processor is configured to send the generated image to the identified second computing device.

3. The medical scan data system of claim 1, wherein the first computing device is a server, and the plurality of second computing devices are client computers.

4. The medical scan data system of claim 1, wherein the first subset of the medical scan data is a first type of medical scan data, and the second subset of the medical scan data is a second type of medical scan data, the first type of medical scan data being different than the second type of medical scan data.

5. The medical scan data system of claim 1, wherein the first subset of the medical scan data is mammography scan data, and the second subset of the medical scan data includes scan data generated by a computed tomography (CT) device, scan data generated by a magnetic resonance imaging (MRI) device, scan data generated by an X-ray device, or any combination thereof.

6. The medical scan data system of claim 1, wherein the identified first subset of the medical scan data is a DICOM image.

7. A method of generating images from medical scan data, the method comprising:
identifying, by a processor in communication with a plurality of computing devices, a first subset of the medical scan data and a second subset of the medical scan data;
identifying, by the processor, a first computing device of the plurality of computing devices based on the identified first subset of the medical scan data;
generating, by the processor, an image based on the second subset of the medical scan data;
sending, by the processor, the identified first subset of the medical scan data to the identified first computing device, and sending the generated image to a second computing device of the plurality of computing devices.

8. The method of claim 7, further comprising:
sending, by the processor, the generated image to the identified first computing device.

9. The method of claim 7, wherein the plurality of computing devices are client computers in communication with a server, the server comprising the processor.

10. The method of claim 7, wherein the first subset of the medical scan data is a first type of medical scan data, and the second subset of the medical scan data is a second type of medical scan data, the first type of medical scan data being different than the second type of medical scan data.

11. An apparatus for generating images from medical scan data, the apparatus comprising:
a first computing device in communication with a second computing device, the first computing device comprising:
a memory configured to store first medical scan data;
a processor in communication with the memory, the processor configured to:
send an identification to the second computing device or another memory;
receive the first medical scan data from the second computing device based on the identification;
receive a request to display a first image and a second image, wherein the first image corresponds to the first medical scan data received by the first computing device and the second image corresponds to second medical scan data stored at the second computing device;
receive the second image from the second computing device;
generate the first image from the first medical scan data; and
a display in communication with the memory and the processor, the display configured to:
display the first image and the second image.

12. The apparatus of claim 11, wherein the identification comprises information about a configuration of the first computing device.

13. The apparatus of claim 11, wherein the first medical scan data is a first type of medical scan data, and the second medical scan data is a second type of medical scan data, the first type of medical scan data being different than the second type of medical scan data.

14. The apparatus of claim 13, wherein the first subset of the medical scan data is mammography scan data, and the second subset of the medical scan data includes scan data generated by a computed tomography (CT) device, scan data generated by a magnetic resonance imaging (MRI) device, scan data generated by an X-ray device, or any combination thereof.

15. The apparatus of claim 11, wherein the identification comprises a request to receive a type of medical scan medical scan data.

16. The apparatus of claim 11, wherein the first computing device is a client, and the second computing device is a server.

17. A method of generating images from medical scan data, the method comprising:
sending, by a processor in communication with a computing device, an identification to the computing device;
receiving, by the processor, first medical scan data from the computing device based on the identification;
receiving, by the processor, a request to display first image and a second image, wherein the first image corresponds to the first medical scan data received by the first computing device and the second image corresponds to second medical scan data stored on the second computing device;
receiving, by the processor, the second image from the computing device;
generating, by the processor, the first image from the first medical scan data; and
displaying the first image and the second image.

18. The method of claim 17, wherein the identification comprises information about the configuration of the processor, devices in communication with the processor, or any combination thereof.

19. The method of claim 17, wherein the first medical scan data is a first type of medical scan data, and the second medical scan data is a second type of medical scan data, the first type of medical scan data being different than the second type of medical scan data.

20. The method of claim 17, wherein the computing device is a server in communication with a client, the client comprising the processor.
